(19) United States
(12) Patent Application Publication
(10) Pub. No.: US 2019/0233836 A1
(43) Pub. Date: Aug. 1, 2019

(54) MEANS AND METHODS TO INCREASE COUMARIN PRODUCTION

(71) Applicants: VIB VZW, Gent (BE); UNIVERSITEIT GENT, Gent (BE)

(72) Inventors: Wout Boerjan, Kalken (BE); Lisa Sundin, Gent (BE); Ruben Vanholme, Destelbergen (BE)

(21) Appl. No.: 16/330,817

(22) PCT Filed: Sep. 6, 2017
(86) PCT No.: PCT/EP2017/072317

§ 371 Date: Mar. 6, 2019

(30) Foreign Application Priority Data
Sep. 6, 2016 (EP) 16187335.1

(51) Int. Cl. C12N 15/82; C12N 9/02

(52) U.S. Cl. CPC C12N 15/8222; C12N 9/0071; C12N 15/8282; C12N 15/8261; C12N 15/8243; C12Y 114/11

(57) ABSTRACT

The present invention relates to the field of plant molecular biology, more particularly to the field of agriculture, even more particularly to the field of improving the yield of coumarins in plants. The present invention provides chimeric genes and constructs which can be used to enhance the coumarin yield.

### FIELD OF THE INVENTION

The present invention relates to the field of plant molecular biology, more particularly to the field of agriculture, even more particularly to the field of improving the yield of coumarins in plants. The present invention provides chimeric genes and constructs which can be used to enhance the coumarin yield in plants and crops.

### INTRODUCTION TO THE INVENTION

Coumarins (1,2-benzopyrones) are a major group of plant secondary metabolites. They play important roles in the environmental adaptation of plants and contribute to the defense against phytopathogens. Coumarin derivatives have demonstrated multiple pharmaceutical activities such as anti-coagulative, anti-fungal and anti-inflammatory actions. In plants, coumarins are synthesized via the general phenylpropanoid pathway. A key step in the formation of coumarin is the ortho-hydroxylation of the aromatic ring of a cinnamic acid by the feruloyl-CoA 6′-hydroxylase 1 (F6′H1). According to the prior art the product 6′-hydroxyferuloyl-CoA is converted into scopoletin (a simple coumarin) via spontaneous trans-cis isomerization and lactonization. In the present invention we have identified a novel enzyme, further designated herein as COSY, which catalyzes the conversion of a range of 6′ hydroxycinnamoyl-CoAs to coumarins such as umbelliferone, esculetin and scopoletin. Importantly, plants lacking the enzyme COSY have a strongly reduced abundance of scopoletin and scopoletin-containing metabolites. In the prior art it is known that plant mutants which are deficient in coumarin biosynthesis suffer from iron deficiency chlorosis when grown in alkaline soils (see Schmid et al, 2014). Indeed, coumarins such as esculetin, scopoletin and fraxetin can contribute to iron uptake either by forming chelates with iron or by increasing its solubility by reducing ferric (Fe³⁺) ion to ferrous (Fe²⁺) ion (Schmid et al, 2014). In the present invention we have shown that plants comprising a chimeric gene expressing COSY and F6′H1 have an increased production of coumarins and can be used to overcome iron deficiency in alkaline soils.

Recombinant plants of the present invention can also be used to confer fungal disease resistance. Indeed it has been shown that plants having an increased expression of F6′H1 and consequently an increased expression of scopoletin have a higher fungal resistance (see WO2016124515). Other uses of the COSY gene and the transgenic plants comprising a chimeric gene comprising COSY are further described herein.

### FIGURE LEGENDS

FIG. 1: Co-expression analysis of COSY with known genes of the lignin biosynthetic pathway. COSY co-expresses with known phenylpropanoid biosynthesis genes (PAL2, C4H, C3H1, CSE) in the background of 9 phenylpropanoid biosynthesis mutants; pall, c4h, 4cl1, 4cl2, ccoaomt1, ccr1, f5h1, comt and cad6.

FIG. 2: Genomic structure and localization of the T-DNA insertions on the COSY gene. Three mutant alleles were isolated. Black boxes, exons; grey box, 5′ and 3′ untranslated regions; P1 to P5, PCR primers used to confirm insert.

FIG. 3: cosy mutants are sensitive to alkaline soils. Three-week-old seedlings of cosy mutants develop chlorosis and necrosis when grown on soil at pH 8.5, while wild-type seedlings were less affected. Watering with 300 μM of Fe-EDDHA (iron with a synthetic iron chelator) largely recovered the phenotype of the mutant and the wild type. Plants grown in short day conditions (9 hour light, 15 hour dark photoperiod).

FIG. 4: Fluorescence Intensity of exudates of four Arabidopsis thaliana p35S:COSY overexpression lines (line 1, 2, 3 and 3.2). The fluorescence intensity of the cosy-3 mutant Ler-0 wild type (wt1) served as control for the p35S:COSY overexpression line 1, whereas the fluorescence intensity of Ler-0 wild type (wt2) served as control for p35S:COSY overexpression lines 2, 3 and 3.2. The data represents the average value of 8 to 9 biological repeats for each line. The error bars depict Standard error (SE). Each repeat consisted of the exudates of 3 seedlings. Significant differences to the mutant are indicated by a different letter at p<0.05 (Student t-test). All plants are from the Landsberg (Ler-0) ecotype.

FIG. 5: Fluorescence Intensity of exudates of A. thaliana pPYK10:F6′H1:T2A:COSY overexpression lines. The data represent the average value of 6 to 8 biological repeats for each line. The error bars depict Standard deviation (SD). Each repeat consisted of the exudates of 3 seedlings. Significant differences are indicated by a different letter, p<0.05 (Student t-test). A) Fluorescence Intensity of exudates of five pPYK10:F6′H1:T2A:COSY overexpression lines (LINE 7.1, 7.4, 7.5, 10.1 and 10.10) in Col-0 background. The cosy-3 mutant is in the Ler-0 background. All pPYK10:F6′H1:T2A:COSY overexpression lines have a significantly higher fluorescence than their wild-type controls (wt col-0). B) Fluorescence Intensity of exudates of five pPYK10:F6′H1:T2A:COSY overexpression lines (line 2.2, 2.3, 2.6, 4.1 and 4.5) in Nössen background. The cosy-1 and cosy-2 are included as controls. Four of the pPYK10:F6′H1:T2A:COSY overexpression lines have a significantly higher fluorescence then their wild-type controls (wt nos), while the fifth line (line 4.5) shows a similar tendency. C) Fluorescence Intensity of exudates of six pPYK10:F6′H1:T2A:COSY overexpression lines (LINE 2.1, 2.3, 3.1, 4.2, 4.5 and 4.6) in Ler-0 background. The cosy-3 is included as control. Five of the pPYK10:F6′H1:T2A:COSY overexpression lines have a significantly higher fluorescence of their root exudates than their wild-type control (Ler-0 WT), while the sixth line (LINE 4.2) shows a similar tendency.

FIG. 6: Accumulation of isoscopoletin and esculin in independent A. thaliana pCESA4:F6′H1:T2A:COSY overexpression lines in three different backgrounds: wild type (Col-0), the ccr1-6 mutant and pSNBE:CCR1 ccr1-6 (vessel). Methanol-soluble phenolics were extracted from inflorescence stems and analyzed using UHPLC-MS. The average peak area of isoscopoletin (grey) and esculin (black) is given in counts. Error bars represent standard deviations. N=5 for Col-0, ccr1-6 and vessel and N>23 for the pCESA4:F6′H1:T2A:COSY overexpression lines. Statistical analysis showed a significant increase in scopoletin and esculin in all pCESA4:F6′H1:T2A COSY overexpression lines as compared to their respective controls (p<0.001).

FIG. 7: Enzymatic activity of COSY. COSY and 4CL were supplemented with either 2-hydroxy-p-coumaric acid (2OHpCA), 6-hydroxycaffeic acid (6OHCA) or 6-hydroxyferulic acid (6OHFA). Umbelliferone, esculetin and scopoletin were formed, respectively, only in the presence of CoA and the enzymes 4CL and COSY. The y-axis represents the normalized abundance, error bars show the standard deviation (n=4).

### DETAILED DESCRIPTION OF THE INVENTION

As used herein, each of the following terms has the meaning associated with it in this section. The articles “a” and “an” are used herein to refer to one or to more than one (i.e., to at least one) of the grammatical object of the article. By way of example, “an element” means one element or more than one element. “About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods. The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments, of the invention described herein are capable of operation in other sequences than described or illustrated herein.

Unless specifically defined herein, all terms used herein have the same meaning as they would to one skilled in the art of the present invention. Practitioners are particularly directed to Sambrook at al., Molecular Cloning: A Laboratory Manual, 4^{th} ed., Cold Spring Harbor Press, Plainsview, N.Y. (2012); and Ausubel et al., current Protocols in Molecular Biology (Supplement 100), John Wiley & Sons, New York (2012), for definitions and terms of the art. The definitions provided herein should not be construed to have a scope less than understood by a person of ordinary skill in the art.

In the present invention we have identified the function of the coding sequence of the gene At1g28680. This gene is according to the public Arabidopsis Information Resource (TAIR database) a HXXXD-type acyl-transferase family protein. Our experimental data show that the function of the gene product of At1g28680 is the enzymatic conversion of the product 2′-hydroxycoumaroyl-CoA, 6′-hydroxycaffeoyl-CoA and 6′-hydroxyferuloyl-CoA into umbelliferone, esculetin and scopoletin via trans/cis isomerization and lactonization. These enzymatic conversions were believed to occur spontaneously. We have designated the new function of the At1g28680 gene product as a coumarine synthesis. In the present invention we further designate this gene and gene product as COSY.

SEQ ID NO: 1 depicts the full length coding sequence of the Arabidopsis thaliana COSY gene (derived from At1g28680):

SEQ ID NO: 2 depicts the protein sequence of Arabidopsis thaliana COSY (At1g28680) Accordingly the present invention provides a chimeric gene construct comprising the following operably linked DNA elements: a) a plant expressible promoter, b) a DNA region encoding SEQ ID NO: 2 or a functional plant orthologue thereof and c) a 3′ end region comprising transcription termination and polyadenylation signals functioning in cells of a plant.

A “chimeric gene” or “chimeric construct” is a recombinant nucleic acid sequence in which a promoter or regulatory nucleic acid sequence is operatively linked to, or associated with, a nucleic acid sequence that codes for an mRNA, such that the regulatory nucleic acid sequence is able to regulate transcription or expression of the associated nucleic acid coding sequence.

The regulatory nucleic acid sequence of the chimeric gene is not operatively linked to the associated nucleic acid sequence as found in nature.

A functional plant orthologue (or a functional plant orthologous gene) of SEQ ID NO: 2 is a plant orthologous gene of COSY which encodes a protein with the same enzymatic properties of COSY, id est the E-Z isomenzation of the side chain of 2′-hydroxycoumaroyl-CoA, 6′-hydroxycaffeoyl-CoA and 6′-hydroxyferuloyl-CoA and lactonization resulting in the products umbelliferone, esculetin and scopoletin. This enzymatic function was first elucidated by the present inventors and several ways to monitor the enzymatic function are described in Example 2.

Functional orthologues COSY genes can be isolated from the (publically) available sequence databases. The “sequence identity” of two related nucleotide or amino acid sequences, expressed as a percentage, refers to the number of positions in the two optimally aligned sequences which have identical residues (×100) divided by the number of positions compared. A gap, i.e., a position in an alignment where a residue is present in one sequence but not in the other is regarded as a position with non-identical residues. The alignment of the two sequences is performed by the Needleman and Wunsch algorithm (Needleman and Wunsch (1970) J Mol Biol. 48: 443-453). The computer-assisted sequence alignment above, can be conveniently performed using standard software program such as GAP which is part of the Wisconsin Package Version 10.1 (Genetics Computer Group, Madision, Wis., USA) using the default scoring matrix with a gap creation penalty of 50 and a gap extension penalty of 3. Sequences are indicated as “essentially similar” when such amino acid sequences have a sequence identity of at least about 75%, particularly at least about 80%, more particularly at least about 85%, quite particularly about 90%, especially about 95%, more especially about 100%, quite especially are identical. Alternatively the skilled person can isolate orthologous plant COSY genes through methods of genetic hybridization. Such methods are well known to the skilled (plant) molecular biologist.

Several examples of plant functional orthologues COSY genes are represented in Example 8.

In yet another embodiment the invention provides a chimeric gene construct comprising the following operably linked DNA elements: a) a plant expressible promoter, b) a multicistronic DNA region encoding SEQ ID NO: 2 or a functional plant orthologue thereof coupled to a DNA region encoding a feruloyl-CoA hydroxylase 1 or a functional plant orthologue thereof and c) a 3′ end region comprising transcription termination and polyadenylation signals functioning in cells of a plant.

The plant feruloyl-CoA 6 [symbol] hydroxylase 1 (F6′H1) catalyzes the ortho-hydroxylation of the aromatic ring of a cinnamic acid. The enzymatic function of F6′H1 is well known to the person skilled in the art (see for example WO2016124515) and functional plant orthologous genes encoding functional F6′H1 can readily be identified by the skilled person.

Functional orthologues F6′H1 genes can be isolated from the (publically) available sequence databases. The “sequence identity” of two related nucleotide or amino acid sequences, expressed as a percentage, refers to the number of positions in the two optimally aligned sequences which have identical residues (×100) divided by the number of positions compared.

A gap, i.e., a position in an alignment where a residue is present in one sequence but not in the other is regarded as a position with non-identical residues. The alignment of the two sequences is performed by the Needleman and Wunsch algorithm (Needleman and Wunsch (1970) J Mol Biol. 48: 443-453). The computer-assisted sequence alignment above, can be conveniently performed using standard software program such as GAP which is part of the Wisconsin Package Version 10.1 (Genetics Computer Group, Madision, Wis., USA) using the default scoring matrix with a gap creation penalty of 50 and a gap extension penalty of 3. Sequences are indicated as “essentially similar” when such amino acid sequences have a sequence identity of at least about 75%, particularly at least about 80%, more particularly at least about 85%, quite particularly about 90%, especially about 95%, more especially about 100%, quite especially are identical. Alternatively the skilled person can isolate orthologous plant F6′H1 genes through methods of genetic hybridization. Such methods are well known to the skilled (plant) molecular biologist.

A representative of a gene encoding this enzyme is depicted in SEQ ID NO: 3. SEQ ID NO: 3 encodes the Arabidopsis thaliana Feruloyl-CoA 6 [symbol] hydroxylase 1, the gene is annotated in TAIR as At3G13610.

SEQ ID NO: 4 is the Arabidopsis thaliana F6′H1 (encoded by SEQ ID NO: 3): The co-expression of multiple genes can be valuable in transgenic plants. To achieve this a multitude of techniques including co-transformation of multiple chimeric genes (here a chimeric gene comprising COSY and another chimeric gene comprising F6′H1, either simultaneously or subsequently), crossing of transgenic plants (commonly known as gene stacking) having (one plant comprising a chimeric gene encoding COSY with another plant comprising a chimeric gene encoding F6′H1), the use of multiple or bidirectional promoters to direct the expression of COSY and F6′H1 on the same construct, the creation of a bicistronic or multicistronic construct wherein F6′H and COSY are operably linked and under control of the same promoter. Multicistronic vectors can be made with IRES elements. However, these elements are quite large (500-600 bp). Alternatives multicistronic vectors are made by using self-cleaving 2A peptides codes between the genes in the multicistronic vector. Examples of commonly used 2A peptides used are T2A, P2A, E2A and F2A.

In a specific embodiment the invention provides a chimeric gene construct comprising the following operably linked DNA elements: a) a plant expressible promoter, b) a bicistronic DNA region encoding SEQ ID NO: 2 or a functional plant orthologue thereof coupled to a DNA region encoding a feruloyl-CoA 6′-hydroxylase 1 or a functional plant orthologue thereof via a T2A sequence and c) a 3′ end region comprising transcription termination and polyadenylation signals functioning in cells of a plant.

An example of a bicistronic construct F6′H1:T2A:COSY is depicted in SEQ ID NO: 5 In yet another embodiment the invention provides a recombinant vector comprising the chimeric gene constructs as described herein before.

The chimeric gene or chimeric genes to be expressed are preferably cloned into a vector, which is suitable for transforming Agrobacterium tumefaciens, for example pBin19 (Bevan et al (1984) Nuc. Acids Res. 12-8711). Agrobacteria transformed by such a vector can then be used in known manner for the transformation of plants, such as plants used as a model, like Arabidopsis (Arabidopsis thaliana is within the scope of the present invention not considered as a crop plant), or crop plants such as, by way of example, tobacco plants, for example by immersing bruised leaves or chopped leaves in an agrobacterial solution and then culturing them in suitable media. The transformation of plants by means of Agrobacterium tumefaciens is described, for example, by Hofgen and Willmitzer in Nuc. Acid Res. (1988) 16, 9877 or is known inter alia from F. F. White, Vectors for Gene Transfer in Higher Plants; in Transgenic Plants, Vol. 1, Engineering and Utilization, eds. S. D. Kung and R. Wu, Academic Press, 1993, pp. 15-38. The term “expression cassette” refers to any recombinant expression system for the purpose of expressing a nucleic acid sequence of the invention in vitro or in vivo, constitutively or inducibly, in any cell, including, in addition to plant cells, prokaryotic, yeast, fungal, insect or mammalian cells. The term includes linear and circular expression systems. The term includes all vectors. The cassettes can remain episomal or integrate into the host cell genome. The expression cassettes can have the ability to self-replicate or not (i.e., drive only transient expression in a cell). The term includes recombinant expression cassettes that contain only the minimum elements needed for transcription of the recombinant nucleic acid. Preferably the vectors comprising the chimeric gene (or genes) of the invention comprise a selectable marker or reporter gene. A “Selectable marker”, “selectable marker gene” or “reporter gene” includes any gene that confers a phenotype on a cell in which it is expressed to facilitate the identification and/or selection of cells that are transfected or transformed with a chimeric gene construct or vector comprising a chimeric gene construct of the invention. These marker genes enable the identification of a successful transfer of the nucleic acid molecules via a series of different principles. Suitable markers may be selected from markers that confer antibiotic or herbicide resistance, that introduce a new metabolic trait or that allow visual selection. Examples of selectable marker genes include genes conferring resistance to antibiotics (such as nptll that phosphorylates neomycin and kanamycin, or hpt, phosphorylating hygromycin, or genes conferring resistance to, for example, bleomycin, streptomycin, tetracyclin, chloramphenicol, ampicillin, gentamycin, geneticin (G418), spectinomycin or blasticidin), to herbicides (for example bar which provides resistance to Basta®; aroA or gox providing resistance against glyphosate, or the genes conferring resistance to, for example, imidazolinone, phosphinothricin or sulfonylurea), or genes that provide a metabolic trait (such as manA that allows plants to use mannose as sole carbon source or xylose isomerase for the utilisation of xylose, or antinutritive markers such as the resistance to 2-deoxyglucose). Expression of visual marker genes results in the formation of colour (for example β-glucuronidase, GUS or β-galactosidase with its coloured substrates, for example X-Gal), luminescence (such as the luciferinluciferase system) or fluorescence (Green Fluorescent Protein, GFP, and derivatives thereof). This list represents only a small number of possible markers. The skilled worker is familiar with such markers. Different markers are preferred, depending on the plant and the selection method. It is known that upon stable or transient integration of nucleic acids into plant cells, only a minority of the cells takes up the foreign DNA and, if desired, integrates it into its genome, depending on the expression vector used and the transfection technique used. To identify and select these integrants, a gene coding for a selectable marker (such as the ones described above) is usually introduced into the host cells together with the gene of interest. These markers can for example be used in mutants in which these genes are not functional by, for example, deletion by conventional methods. Furthermore, nucleic acid molecules encoding a selectable marker can be introduced into a host cell on the same vector that comprises the sequence encoding the polypeptides of the invention or used in the methods of the invention, or else in a separate vector. Cells which have been stably transfected with the introduced nucleic acid can be identified for example by selection (for example, cells which have integrated the selectable marker survive whereas the other cells die). Since the marker genes, particularly genes for resistance to antibiotics and herbicides, are no longer required or are undesired in the transgenic host cell once the nucleic acids have been introduced successfully, the process according to the invention for introducing the nucleic acids advantageously employs techniques which enable the removal or excision of these marker genes. One such a method is what is known as co-transformation. The co-transformation method employs two vectors simultaneously for the transformation, one vector bearing the nucleic acid according to the invention and a second bearing the marker gene(s). A large proportion of transformants receives or, in the case of plants, comprises (up to 40% or more of the transformants), both vectors. In case of transformation with Agrobacteria, the transformants usually receive only a part of the vector, i.e. the sequence flanked by the T-DNA, which usually represents the expression cassette. The marker genes can subsequently be removed from the transformed plant by performing crosses. In another method, marker genes integrated into a transposon are used for the transformation together with desired nucleic acid (known as the Ac/Ds technology). The transformants can be crossed with a transposase source or the transformants are transformed with a nucleic acid construct conferring expression of a transposase, transiently or stable. In some cases (approx. 10%), the transposon jumps out of the genome of the host cell once transformation has taken place successfully and is lost. In a further number of cases, the transposon jumps to a different location. In these cases the marker gene must be eliminated by performing crosses. In microbiology, techniques were developed which make possible, or facilitate, the detection of such events. A further advantageous method relies on what is known as recombination systems; whose advantage is that elimination by crossing can be dispensed with. The best-known system of this type is what is known as the Cre/lox system. Cre1 is a recombinase that removes the sequences located between the loxP sequences. If the marker gene is integrated between the loxP sequences, it is removed once transformation has taken place successfully, by expression of the recombinase. Further recombination systems are the HIN/HIX, FLP/FRT and REP/STB system (Tribble et al., J. Biol. Chem., 275, 2000: 22255-22267; Velmurugan et al., J. Cell Biol., 149, 2000: 553-566). A site-specific integration into the plant genome of the nucleic acid sequences according to the invention is possible.

For the purposes of the invention, “transgenic”, “transgene” or “recombinant” means with regard to, for example, a nucleic acid sequence, an expression cassette, chimeric gene construct or a vector comprising the nucleic acid sequence or an organism transformed with the nucleic acid sequences, expression cassettes or vectors according to the invention.

In yet another particular embodiment the invention provides a plant, plant cell or plant seed comprising a chimeric gene construct or chimeric gene constructs described herein before or comprising a recombinant vector comprising a chimeric gene construct of the invention.

In yet another embodiment the invention provides a plant, plant cell or plant seed comprising a chimeric gene construct comprising the following operably linked DNA elements: a) a plant expressible promoter, b) a DNA region encoding SEQ ID NO: 2 or a functional plant orthologue thereof and c) a 3′ end region comprising transcription termination and polyadenylation signals functioning in cells of a plant further comprising a second chimeric gene construct comprising the following operably linked DNA elements: a) a plant expressible promoter, b) a DNA region encoding a feruloyl-CoA 6 [symbol] hydroxylase 1 or a functional plant orthologue thereof and c) a 3′ end region comprising transcription termination and polyadenylation signals functioning in cells of a plant.

In the present invention a “plant promoter” comprises regulatory elements, which mediate the expression of a coding sequence segment in plant cells. For expression in plants, the nucleic acid molecule must be linked operably to or comprise a suitable promoter which expresses the gene at the right point in time and with the required spatial expression pattern.

The term “operably linked” as used herein refers to a functional linkage between the promoter sequence and the gene of interest, such that the promoter sequence is able to initiate transcription of the gene of interest.

A “constitutive promoter” refers to a promoter that is transcriptionally active during most, but not necessarily all, phases of growth and development and under most environmental conditions, in at least one cell, tissue or organ. The term “terminator” encompasses a control sequence which is a DNA sequence at the end of a transcriptional unit which signals 3′ processing and polyadenylation of a primary transcript and termination of transcription. The terminator can be derived from the natural gene, from a variety of other plant genes, or from T-DNA. The terminator to be added may be derived from, for example, the nopaline synthase or octopine synthase genes, or alternatively from another plant gene, or less preferably from any other eukaryotic gene.

In the chimeric genes of the invention the promoter can be constitutive promoter (e.g. 35S promoter), pathogen inducible promoter, fungal inducible, mesophyll-specific promoter and/or epidermis—specific promoter, stalk-specific promoter, ear-specific promoter or kernel-specific promoter.

In a specific embodiment the promoter is a root specific promoter.

An example of a strong and specific root specific promoter is the PYK10 promoter (see Example 10 for the sequence of the Arabidopsis thaliana PYK10 promoter). Other root-specific promoters are promoters like for example GmPRP2 and GmTIP and still other root specific promoters disclosed in Chen et al (2014) and Chen et al (2015).

In yet another specific embodiment in the chimeric genes of the invention a promoter expressed in the plant secondary cell wall can be used. An example of such a promoter is derived from the plant CESA4 gene. The sequence of the Arabidopsis CESA4 promoter is depicted in Example 10. Still other examples of promoters derived from genes involved in cellulose biosynthesis of the secondary cell wall are those of the CESA7 and CESA8 genes in A. thaliana.

In yet another embodiment in the chimeric genes of the invention a promoter involved in lignin biosynthesis van be used. Non-limiting examples of the promoters of genes involved in lignin biosynthesis are PAL, C4H, 4CL, HCT, C3H, CSE, CCoAOMT, CCR1, F5H, COMT and CAD or non-Arabidopsis plant orthologues genes thereof.

For the identification of functionally equivalent plant root specific promoters (for example in other plant genera or other plant species), the promoter strength and/or expression pattern of a candidate root specific promoter (for example the Arabidopsis PYK10 promoter) may be analysed for example by operably linking the promoter to a reporter gene and assaying the expression level and pattern of the reporter gene in the plant. Suitable well-known reporter genes include for example beta-glucuronidase; beta-galactosidase or any fluorescent protein. The promoter activity is assayed by measuring the enzymatic activity of the beta-glucuronidase or beta-galactosidase. Alternatively, promoter strength may also be assayed by quantifying mRNA levels or by comparing mRNA levels of the nucleic acid, with mRNA levels of housekeeping genes such as 18S rRNA, using methods known in the art, such as Northern blotting with densitometric analysis of autoradiograms, quantitative real-time PCR or RT-PCR (Heid et al., 1996 Genome Methods 6: 986-994).

A transgenic plant for the purposes of the invention is thus understood as meaning, as above, that the nucleic acids used in the method of the invention are not present in, or originating from, the genome of said plant, or are present in the genome of said plant but not at their natural locus in the genome of said plant, it being possible for the nucleic acids to be expressed homologously or heterologously. However, as mentioned, transgenic also means that, while the nucleic acids according to the invention or used in the inventive method are at their natural position in the genome of a plant, the sequence has been modified with regard to the natural sequence, and/or that the regulatory sequences of the natural sequences have been modified. Transgenic is preferably understood as meaning the expression of the nucleic acids according to the invention at an unnatural locus in the genome, i.e. homologous or, heterologous expression of the nucleic acids takes place. Preferred transgenic plants are mentioned herein.

The term “introduction” or “transformation” as referred to herein encompass the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated there from. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid. Alternatively, it may be integrated into the host genome. The resulting transformed plant cell may then be used to regenerate a transformed plant in a manner known to persons skilled in the art.

The transfer of foreign genes into the genome of a plant is called transformation. Transformation of plant species is now a fairly routine technique. Advantageously, any of several transformation methods may be used to introduce the gene of interest into a suitable ancestor cell. The methods described for the transformation and regeneration of plants from plant tissues or plant cells may be utilized for transient or for stable transformation. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts (Krens, F. A. et al., (1982) Nature 296, 72-74; Negrutiu I et al. (1987) Plant Mol Biol 8: 363-373); electroporation of protoplasts (Shillito R. D. et al. (1985) Bio/Technol 3, 1099-1 102); microinjection into plant material (Crossway A et al., (1986) Mol. Gen Genet 202: 179-185); DNA or RNA-coated particle bombardment (Klein T M et al., (1987) Nature 327: 70) infection with (non-integrative) viruses and the like. Transgenic plants, including transgenic crop plants, are preferably produced via Agrobacterium-mediated transformation. An advantageous transformation method is the transformation in planta. To this end, it is possible, for example, to allow the agrobacteria to act on plant seeds or to inoculate the plant meristem with agrobacteria. It has proved particularly expedient in accordance with the invention to allow a suspension of transformed agrobacteria to act on the intact plant or at least on the flower primordia. The plant is subsequently grown on until the seeds of the treated plant are obtained (Clough and Bent, Plant J. (1998) 16, 735-743). Methods for Agrobacterium-mediated transformation of rice include well known methods for rice transformation, such as those described in any of the following: European patent application EP1198985, Aldemita and Hodges (Planta 199: 612-617, 1996); Chan et al. (Plant Mol Biol 22 (3): 491-506, 1993), Hiei et al. (Plant J 6 (2): 271-282, 1994), which disclosures are incorporated by reference herein as if fully set forth. In the case of corn transformation, the preferred method is as described in either Ishida et al. (Nat. Biotechnol 14(6): 745-50, 1996) or Frame et al. (Plant Physiol 129(1): 13-22, 2002), which disclosures are incorporated by reference herein as if fully set forth. Said methods are further described by way of example in B. Jenes et al., Techniques for Gene Transfer, in: Transgenic Plants, Vol. 1, Engineering and Utilization, eds. S. D. Kung and R. Wu, Academic Press (1993) 128-143 and in Potrykus Annu. Rev. Plant Physiol. Plant Molec. Biol. 42 (1991) 205-225). The nucleic acids or the construct to be expressed is preferably cloned into a vector, which is suitable for transforming Agrobacterium tumefaciens, for example pBin19 (Bevan et al (1984) Nucl. Acids Res. 12-8711). Agrobacteria transformed by such a vector can then be used in known manner for the transformation of plants, such as plants used as a model, like Arabidopsis (Arabidopsis thaliana is within the scope of the present invention not considered as a crop plant), or crop plants such as, by way of example, tobacco plants, for example by immersing bruised leaves or chopped leaves in an agrobacterial solution and then culturing them in suitable media. The transformation of plants by means of Agrobacterium tumefaciens is described, for example, by Hofgen and Winlmitzer in Nucl. Acid Res. (1988) 16, 9877 or is known inter alia from F. F. White, Vectors for Gene Transfer in Higher Plants; in Transgenic Plants, Vol. 1, Engineering and Utilization, eds. S. D. Kung and R. Wu, Academic Press, 1993, pp. 15-38.

In addition to the transformation of somatic cells, which then have to be regenerated into intact plants, it is also possible to transform the cells of plant meristems and in particular those cells which develop into gametes. In this case, the transformed gametes follow the natural plant development, giving rise to transgenic plants. Thus, for example, seeds of Arabidopsis are treated with agrobacteria and seeds are obtained from the developing plants of which a certain proportion is transformed and thus transgenic [Feldman, K A and Marks M D (1987). Mol Gen Genet 208:1-9; Feldmann K (1992). In: C Koncz, N-H Chua and J Shell, eds, Methods in Arabidopsis Research. Word Scientific, Singapore, pp. 274-289]. Alternative methods are based on the repeated removal of the inflorescences and incubation of the excision site in the center of the rosette with transformed agrobacteria, whereby transformed seeds can likewise be obtained at a later point in time (Chang (1994). Plant J. 5: 551-558; Katavic (1994). Mol Gen Genet, 245: 363-370). However, an especially effective method is the vacuum infiltration method with its modifications such as the “floral dip” method. In the case of vacuum infiltration of Arabidopsis, intact plants under reduced pressure are treated with an agrobacterial suspension [Bechthold, N (1993). CR Acad Sci Paris Life Sci, 316: 1 194-1 199], while in the case of the “floral dip” method the developing floral tissue is incubated briefly with a surfactant-treated agrobacterial suspension [Clough, S J and Bent A F (1998) The Plant J. 16, 735-743]. A certain proportion of transgenic seeds are harvested in both cases, and these seeds can be distinguished from non-transgenic seeds by growing under the above-described selective conditions. In addition the stable transformation of plastids is of advantages because plastids are inherited maternally is most crops reducing or eliminating the risk of transgene flow through pollen. The transformation of the chloroplast genome is generally achieved by a process which has been schematically displayed in Klaus et al., 2004 [Nature Biotechnology 22 (2), 225-229]. Briefly the sequences to be transformed are cloned together with a selectable marker gene between flanking sequences homologous to the chloroplast genome. These homologous flanking sequences direct site specific integration into the plastome. Plastidal transformation has been described for many different plant species and an overview is given in Bock (2001) Transgenic plastids in basic research and plant biotechnology. J Mol Biol. 2001 September 21; 312 (3):425-38 or Maliga, P (2003) Progress towards commercialization of plastid transformation technology. Trends Biotechnol. 21, 20-28. Further biotechnological progress has recently been reported in form of marker free plastid transformants, which can be produced by a transient co-integrated maker gene (Klaus et al., 2004, Nature Biotechnology 22(2), 225-229).

The genetically modified plant cells can be regenerated via all methods with which the skilled worker is familiar. Suitable methods can be found in the abovementioned publications by S. D. Kung and R. Wu, Potrykus or Hofgen and Willmitzer.

Generally after transformation, plant cells or cell groupings are selected for the presence of one or more markers which are encoded by plant-expressible genes co-transferred with the gene of interest, following which the transformed material is regenerated into a whole plant. To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Alternatively, the transformed plants are screened for the presence of a selectable marker such as the ones described above.

Following DNA transfer and regeneration, putatively transformed plants may also be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

The terms “increase”, “improve” or “enhance” are interchangeable and shall mean in the sense of the application at least a 3%, 4%, 5%, 6%, 7%, 8%, 9% or 10%, preferably at least 15% or 20%, more preferably 25%, 30%, 35%, 40%, 50%, 60%, 70%, 80%, 90%, 95% or more e.g. coumarin production or fungal disease resistance in comparison to control plants as defined herein.

In yet another embodiment the invention provides the use of a chimeric gene or chimeric genes of the present invention or a recombinant vector comprising a chimeric gene or chimeric genes of the invention to prevent iron deficiency chlorosis in plants.

In yet another embodiment the invention provides the use of a chimeric gene or chimeric genes of the present invention or a recombinant vector comprising a chimeric gene or chimeric genes of the invention to increase the production of coumarins in plants.

In yet another embodiment the invention provides the use of a chimeric gene or chimeric genes of the present invention or a recombinant vector comprising a chimeric gene or chimeric genes of the invention to increase fungal resistance in plants.

In yet another embodiment the invention provides the use of a chimeric gene or chimeric genes of the present invention or a recombinant vector comprising a chimeric gene or chimeric genes of the invention to produce alternative lignin monomers in plants.

A method for producing a plant with reduced iron deficiency chlorosis compared to a corresponding wild type plant, whereby the method comprises introducing or transforming a plant with a chimeric gene or chimeric genes of the present invention or a recombinant vector comprising a chimeric gene or chimeric genes of the invention and selecting a plant with a stable expression of said chimeric gene.

A method for producing a plant with increased coumarin production compared to a corresponding wild type plant, whereby the method comprises introducing or transforming a plant with a chimeric gene or chimeric genes of the present invention or a recombinant vector comprising a chimeric gene or chimeric genes of the invention and selecting a plant with a stable expression of said chimeric gene.

A method for producing a plant with increased fungal resistance compared to a corresponding wild type plant, whereby the method comprises introducing or transforming a plant with a chimeric gene or chimeric genes of the present invention or a recombinant vector comprising a chimeric gene or chimeric genes of the invention and selecting a plant with a stable expression of said chimeric gene.

A method for producing a plant with alternative lignin monomers compared to a corresponding wild type plant which lacks said lignin monomers, whereby the method comprises introducing or transforming a plant with a chimeric gene or chimeric genes of the present invention or a recombinant vector comprising a chimeric gene or chimeric genes of the invention.

The term “plant” as used herein encompasses whole plants, ancestors and progeny of the plants and plant parts, including seeds, shoots, stems, leaves, roots (including tubers), flowers, and tissues and organs, wherein each of the aforementioned comprise the gene/nucleic acid of interest. The term “plant” also encompasses plant cells, suspension cultures, callus tissue, embryos, meristematic regions, gametophytes, sporophytes, pollen and microspores, again wherein each of the aforementioned comprises the gene/nucleic acid of interest.

Plants that are particularly useful in the methods of the invention include in particular monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Alium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula syvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybemum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp., amongst others.

The choice of suitable control plants is a routine part of an experimental setup and may include corresponding wild type plants or corresponding plants without the gene of interest. The control plant is typically of the same plant species or even of the same variety as the plant to be assessed. The control plant may also be a nullizygote of the plant to be assessed. Nullizygotes are individuals missing the transgene by segregation. A “control plant” as used herein refers not only to whole plants, but also to plant parts, including seeds and seed parts.

The following non-limiting Examples describe methods and means according to the invention. Unless stated otherwise in the Examples, all techniques are carried out according to protocols standard in the art. The following examples are included to illustrate embodiments of the invention. Those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### EXAMPLES

1. Characterization of the Arabidopsis thaliana COSY Mutants

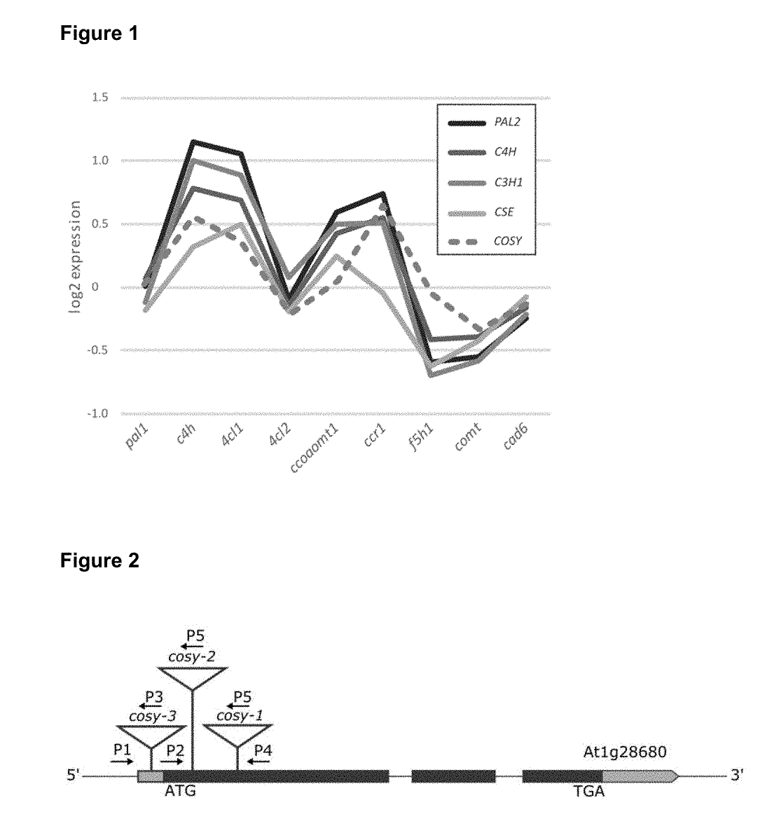

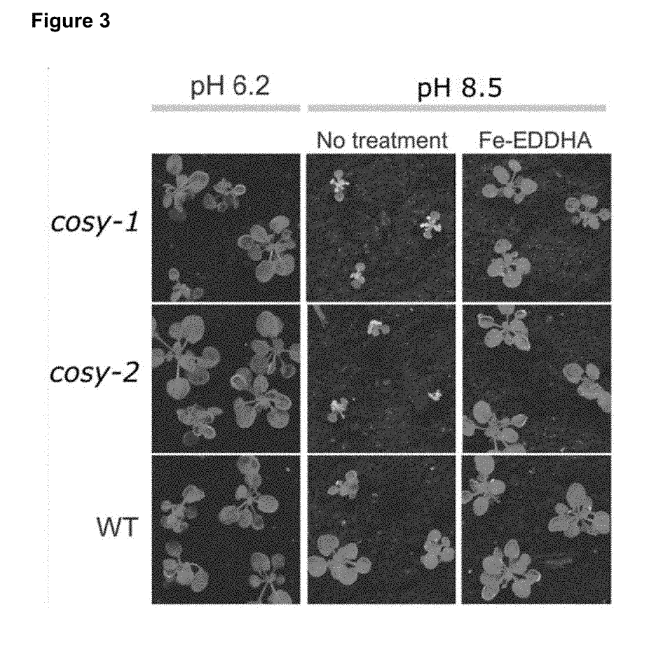

We performed an extensive co-expression analysis using previously generated transcriptome datasets obtained from stems of mutants perturbed in 10 genes of the general phenylpropanoid and monolignol biosynthetic pathways (see Vanholme et al., 2012). In this way, we identified At1g28680, which we have designated COSY here, as a candidate to be involved in the phenylpropanoid or a closely related pathway (FIG. 1). To study the function of COSY, three cosy mutant alleles were obtained. Two T-DNA insertion lines, cosy-1 (RATM15-5543-1) and cosy-2 (RATM13-3585) in ecotype Nössen (AC line), were acquired from the RIKEN Arabidopsis Ds transposon mutant collection (Kuromori et al., 2004) and a third T-DNA insertion line, cosy-3 (GT_3_9907) in ecotype Landsberg erecta (Ler-0), was acquired from the EXON Trapping Insert Consortium (EXOTIC) (Sundaresan at al., 1995). The T-DNA insertion sites were determined by PCR. cosy-1 and cosy-2 have an insertion in the first exon whereas that of cosy-3 is in the 5′-UTR (FIG. 2). Methanol-soluble phenolics were extracted from complete root systems of 3-month-old soil-grown cosy-1 and cosy-2, and wild-type plants. Of the 24 characterized compounds that were more abundant in the cosy mutants, the majority were ortho-hydroxylated phenylpropanoids. These could be further divided into 7 subclasses: 2-hydroxy-p-coumaric acid derivatives, 6-hydroxyferulic acid derivatives, 6-hydroxyconiferyl alcohol derivatives, 6-hydroxydihydroferulic acid derivatives, dihydroscopoletin derivatives, scopoletin derivatives, and others. Of the 17 characterized compounds that were reduced in abundance in cosy-1 and cosy-2, all but one compound (3-methylbutyl glucosinolate) were derivatives of the coumarin scopoletin. This list included various combinations of scopoletin coupled to moieties such as pentose, hexose, sulfate, malonate, ferulate, sinapate and coniferyl alcohol. In addition, two isomers of hydroxy-methoxyscopoletin acetylhexoside, i.e., another type of coumarin, were lower in abundance. Coumarins fluoresce blue when exposed to UV light with a wavelength of 365 nm (Ahn et al, 2010). In agreement with the fact that the abundance of a set of scopoletin-containing metabolites is strongly reduced in the roots of cosy mutants as compared to WT, the roots of all three in vitro grown cosy mutants showed reduced blue fluorescence. Also the medium in the proximity of the root was less fluorescent, as witnessed after removing the plants from the plates. Mutants deficient in the biosynthesis of coumarins or the excretion of coumarins to the rhizosphere show symptoms of iron deficiency such as interveinal chlorosis when grown at elevated pH (Schmid et al., 2014, Schmidt et al., 2014). This chlorosis has been attributed to insufficient iron uptake that results in reduced levels of chlorophyll (Abadia et al., 1999). To examine whether cosy mutants also develop interveinal chlorosis at elevated soil pH, the phenotypes of all three cosy mutants and the wild type were compared when grown on soil with regular pH (pH 6.2) and soil with pH 8.5. On soil of pH 8.5, all three mutants were severely chlorotic, starting from the first pair of true leaves, whereas the wild type only showed minor growth retardation (FIG. 3). After 4 weeks of growth on pH 8.5 soil, cosy-1 and cosy-2 mutants developed necrosis and they did not survive longer than 6 weeks. The wild type did not develop necrosis. The cosy-3 mutant was also chlorotic but grew better compared to cosy-1 and cosy-2. By watering the plants with the synthetic chelator complex Fe-EDDHA, starting immediately after germination, the cosy mutants did not develop the chlorotic phenotype and grew almost equally well as the wild type on soil of pH 8.5 (FIG. 3). Altogether, these results suggest that cosy mutants fail to acquire sufficient iron to sustain photosynthesis.

### 2. Enzymatic Activity of COSY

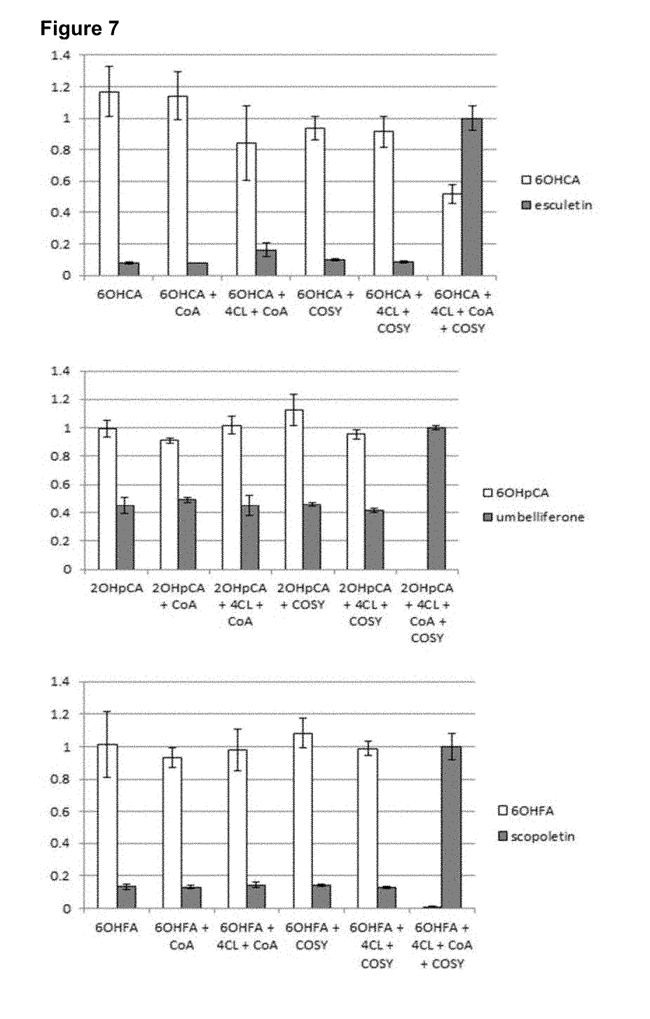

Although the idea of spontaneous isomerization and lactonisation in the biosynthesis of coumarins is now widely accepted in literature (Matsumoto S. et al., 2012; Yang et al., 2015) our cosy mutant data in Example 1 clearly contradict this. When we took a closer examination at the lactonisation reaction of the coumarin core structures we investigated the possible resemblance to an esterification-type reaction mediated by BAHD acyltransferases. BAHD family members utilize activated CoA-thioesters as acyl-donors and an alcohol or amine as the acyl acceptor to form an ester or amide, respectively. We therefore hypothesized that COUMARIN SYNTHASE (COSY) could be a BAHD acyltransferase responsible for the isomerization and lactonization of ortho-hydroxy-cinnamoyl CoA thioesters into coumarins. To verify the catalytic activity of COSY in vitro, COSY was expressed in Escherichia coli and the corresponding COSY protein purified. Potential substrates were chosen based on the phenolic profiling of the cosy mutants. In addition, the fact that all characterized enzymes of the BAHD family utilize CoA-thioesters as acyl donors and the majority uses an alcohol as the acyl acceptor, suggested that 2′-hydroxy-p-coumaroyl-CoA and 6′-hydroxyferuloyl-CoA were the substrates for COSY in A. thaliana. Because neither 2′-hydroxy-p-coumaroyl-CoA, 6′-caffeoyl-CoA nor 6′-hydroxyferuloyl-CoA were commercially available, the corresponding acids were synthesized and the enzymatic assays were performed in combination with a tobacco 4-COUMARATE:CoA LIGASE (4CL) to catalyze the formation of the corresponding CoA thioesters in situ (Beuerle & Pichersky, 2002). In the samples where 4CL and COSY were added simultaneously, seemingly 100% 2-hydroxy-p-coumaric acid and 6-hydroxyferulic acid were converted into the corresponding coumarins, i.e., umbelliferone and scopoletin. About 50% of the 6-hydroxycaffeic acid was converted to the esculetin (see FIG. 7), demonstrating that 4CL is able to attach the CoA to the ortho-hydroxycinnamic acids and that COSY is able to catalyze the reaction forming both umbelliferone, esculetin and scopoletin. The supplemented 2-hydroxy-p-coumaric acid contained the trans and cis isomers, both of which were converted to umbelliferone. The 6-hydroxyferulic acid sample contained only the trans isomer, showing that the COSY enzyme is able to catalyze both the trans-cis isomerization and the subsequent lactonisation. The background scopoletin, esculetin and umbelliferone levels, correspond to impurities of coumarin levels present in the ortho-hydroxycinnamic acid chemical standards used as substrates.

### 3. Plants Comprising a Chimeric Gene Comprising COSY

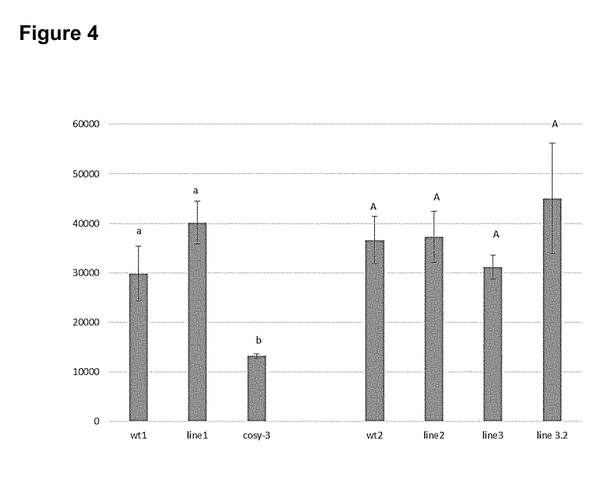

A. thaliana (Ler-0) plants with the chimeric gene p35S:COSY were made to overexpress COSY. Scopoletin levels in root exudates of those plants were measured via fluorescence. Arabidopsis seeds were sterilized and sown on 0.5× Murashige and Skoog agar plates (pH 5.7, 0.8% agar). Seeds were stratified for 48 hours at 4° C. and transferred to the growth chamber. After 3 days, seedlings were placed in 96 well plates containing liquid MS media. 3 seedlings were placed per well and the plate was put on the shaker for 5 days. The seedlings were then removed from the plate and 50 μL of the liquid media taken to measure the fluorescence intensity on a spectrofluorimeter [Perkin Elmer; excitation filter with a central wavelength (CWL)=355 nm and a bandwidth (BW)=40 nm; emission filter with a CWL=460 nm and BW=25 nm]. There was only a small difference between the fluorescence of the p35S:COSY lines and their corresponding wild-type control (FIG. 4). Therefore, we hypothesized that the 35S promoter operably linked with COSY is not optimal to overproduce scopoletin, at least for the A. thaliana ecotype Ler-0. A significant difference was observed between the exudate fluorescence of the cosy-3 mutant and that of its corresponding wild-type control, again showing that COSY is involved in scopoletin biosynthesis. This result is similar to the lower fluorescence of root-exudates of the f67h1 mutant, which has lower scopoletin levels (Schmid et al., 2014).

### 4. Plants Comprising a Chimeric Bicistronic Construct Comprising F6′H1 and COSY in Roots

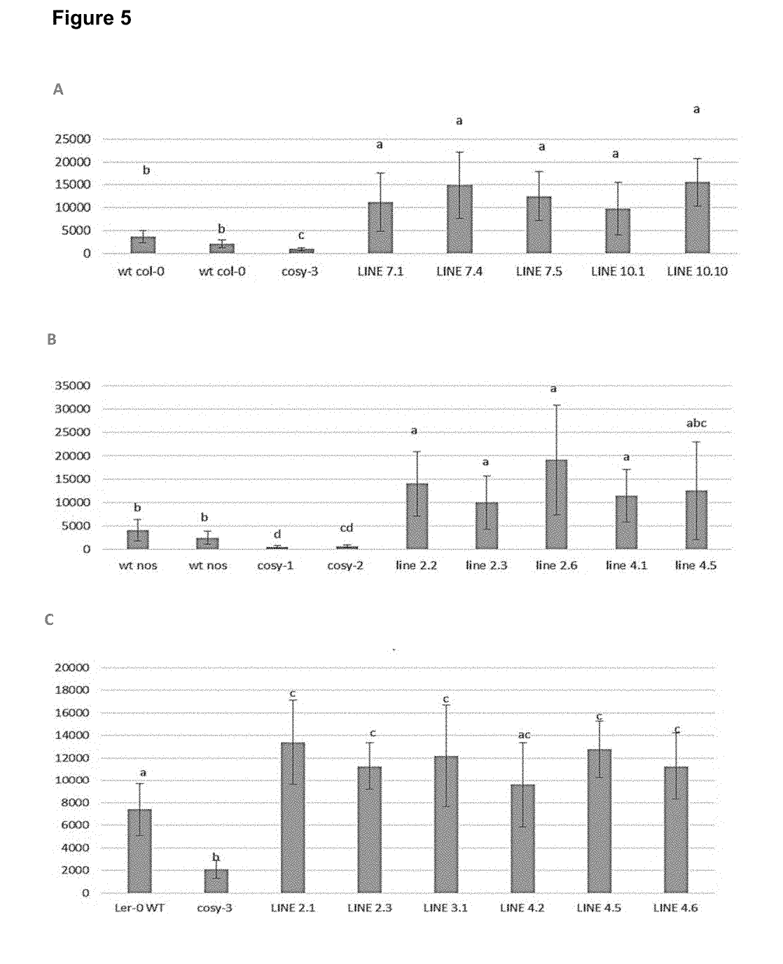

A. thaliana plants with the chimeric gene pPYK10:F6′H1:T2A:COSY were made to overexpress both F6′H1 and COSY in a root-specific manner. Three different ecotypes of A. thaliana were used: Nössen, Landsberg erecta (Ler-0) and Columbia (Col-0). The fluorescence of their root exudates was measured as described above. In all 3 ecotypes there was a significant increase in fluorescence in pPYK10:F6′H1:T2A:COSY over-expression lines as compared to their respective wild-type controls (FIG. 5). These results show that the pPYK10:F671:2A:COSY construct is able to overproduce scopoletin in three different ecotypes of A. thaliana, when grown in liquid MS media (pH 5.7).

5. Plants which Overexpress F6′H and COSY Throughout the Plant

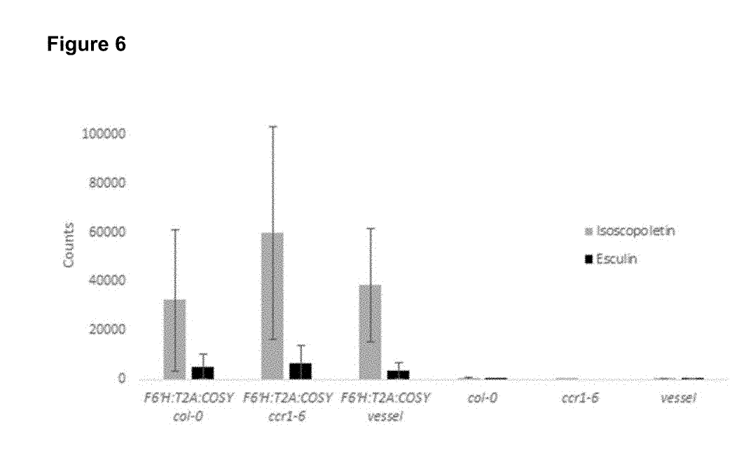

A. thaliana plants with the chimeric gene pCESA4:F67-1:T2A:COSY were generated to overexpress both F6′H1 and COSY in cells that develop secondary cell walls. Three different A. thaliana backgrounds were used to evaluate the effect of simultaneous ectopic overexpression of F6′H1 and COSY. The chimeric gene pCESA4:F6′H1:T2A:COSY was transformed in the Columbia (Col-0) background, in ccr1-6 mutants lacking a functional CINNAMOYL-COA REDUCTASE, and in vessel complemented ccr1-6 mutant (pSNBE:CCR1 ccr1-6) (McCarthy et al., 2011). Phenolic profiling of multiple independent lines revealed a significant increase in isoscopoletin and esculin levels in all backgrounds overexpressing pCESA4:F6′H1:T2A:COSY (FIG. 6). Similarly, a statistically significant increase in levels of other coumarins (among which, scopoletin, skimmin and scopolin) was observed (p<0.001; data not shown). In addition, all metabolite extracts obtained from F6′H1 and COSY overexpressing lines showed an elevated fluorescence under UV light, demonstrating an increase in the total coumarin content of the samples.

### 6. Conditions to Test Poplar Overexpressing F6′H1 and COSY

Recombinant poplars comprising the chimeric construct pPYK10:F6′H1:T2A:COSY are grown on alkali soil of pH 7.5 (calcium carbonate and sodium bicarbonate at 3.43 g/kg of soil and 25.7 g/kg of soil, respectively). It is well known that the reduction component in the iron-uptake strategy I is hindered by high pH when the protons are buffered by bicarbonates, e.g., in calcareous soils (Ohwaki &Sugahara, 1997) and indeed wild-type poplars (Populus tremula×alba) are chlorotic when grown on pH 7.5. Lines that produce scopoletin to a higher extend than wild-type poplars, will have improved iron uptake and thus reduced chlorosis. Since iron deficiency is readily visible due to the distinct interveinal chlorosis we visually monitor the pPYK10:F6′H1:T2A:COSY over-expression and wild-type lines by observing the phenotype of the leaves. In addition, the iron and chlorophyll content in the leaves and the biomass will be measured as a way of quantitatively assessing how over expression lines have improved iron uptake. The growth performance is measured by determining the height and mass of the plants.

7. Use of Plants with Increased Coumarin Biosynthesis

A first important role for coumarins in non-graminaceous monocot and dicot plants (strategy I plants) is iron uptake. Coumarins such as esculetin, scopoletin and fraxetin can contribute to iron uptake either by forming chelates with iron or by increasing its solubility by reducing ferric (Fe³⁺) ion to ferrous (Fe²⁺) ion (Schmid et al., 2014, Schmidt et al., 2014, Rodriguez-Celma at al., 2013). Iron deficiency in plants especially occurs on alkaline soils where high concentrations of calcium carbonate or calcium sulfate ions are present. Iron deficient plants show chlorosis, i.e. yellowing of the leaves, and are affected in the biomass yield. Thus, plants with increased coumarin biosynthesis should aid in iron uptake and thus improve the yield, especially on alkaline soils.

Second, coumarins (including scopoletin) are considered as phytoalexins because of their antimicrobial and anti-insect properties. For instance, down regulation of F6′H1 in tobacco resulted in reduced scopoletin levels together with an increased susceptibility towards Alternaria alternata (Sun et al, 2014). Moreover, antisense down regulation of a tobacco glycosyltransferase results in a reduction of scopoletin and a deceased resistance against the tobacco mosaic virus (Chong, 2002). Thus, plants with increased coumarin biosynthesis should be more resistant against specific pathogens including bacteria (e.g. Alternaria alternate, Ralstonia solanacearum, Phytophthora palmivora) and fungi (Ophiostoma ulmi, Fusarium oxysporum) and insects (e.g. Spilartctia oblique). This is also described in WO2016124515.

Third, coumarins that are hydroxylated on the aromatic ring (such as umbelliferone, esculetin, scopoletin, fraxetin and umckalin) might act as alternative lignin monomers. Alternative lignin monomers are monomers that are rare or absent in lignin of wild-type plants and they are termed ‘alternative monolignols’ to differentiate them from the traditional monolignols (p-coumaryl, coniferyl and sinapyl alcohol) that do make up the bulk of lignin in wild-type plants (Vanholme et al., 2012). Introducing coumarins (such as umbelliferone, esculetin, scopoletin, fraxetin and umckalin) in the lignin polymer could result in lignin with improved properties in terms of (industrial) degradability or down-stream processing of lignin in renewable materials, while it should not affect plant growth and development.

### 8. Use of COSY in White Biotech Applications

Several coumarins can be obtained via extraction from plant resources. Nevertheless, the use of microorganisms engineered to synthesize coumarins could be a valuable alternative to obtain coumarins, which can be used directly or are further processed via synthesis of coumarin derivatives. For instance, Yang et al (2015) teach a process to synthesize coumarins by feeding phenylpropanoids to Escherichia coli expressing two genes: 4-COUMARATE COA:LIGASE (4CL) and F6′H. Since we show here that the synthesis pathway towards coumarins is not complete without COSY. The introduction of COSY in such a microorganism could improve coumarin yields considerably.

### 9. Orthologous Functional Plant COSY Genes

SEQ ID NO: 6: Populus trichocarpa COSY (PT04G05350)
SEQ ID NO: 7: Protein sequence Populus trichocarpa COSY (PT04G05350)

### SEQ ID NO: 8: Glycine max COSY (GM13G30550)

SEQ ID NO: 9: Protein sequence Glycine max COSY (GM13G30550)
SEQ ID NO: 10: Full length coding sequence Medicago truncatula COSY (MT2G015430)
SEQ ID NO: 11: Protein sequence Medicago truncatula COSY (MT2G015430)
SEQ ID NO: 12: Oryza sativa ssp. japonica COSY (OS06G06180)
SEQ ID NO: 13: Protein sequence Oryza sativa ssp. japonica COSY (OS06G06180)

### 10. Promoter Sequences Used in the Invention

SEQ ID NO: 14: Promoter sequence of Arabidopsis thaliana PYK10 (AT3G09260)
SEQ ID NO: 15: Promoter sequence of Arabidopsis thaliana CESA4 (At5g44030)
SEQ ID NO: 16: Promoter sequence of cauliflower mosaic virus (CaMV) 35S

### REFERENCES

Abadia J, Morales F, Abadia A, 1999. Photosystem II efficiency in low chlorophyll, iron-deficient leaves. Plant and Soil 215, 183-92.
Ahn Y O, Shimizu B, Sakata K, et al., 2010. Scopolin-hydrolyzing beta-glucosidases in roots of Arabidopsis. Plant Cell Physiol 51, 132-43.
Beuerle T, Pichersky E, 2002. Enzymatic synthesis and purification of aromatic coenzyme a esters. Anal Biochem 302, 305-12.
Chen, L., Jiang, B., Wu, C., Sun S., Hou W., Han T. (2014) GmPRP2 promoter drives root-preferential expression in transgenic Arabidopsis and soybean hairy roots BMC Plant Biology, 14:245
Chen, L., Jiang, B., Wu, C., Sun S., Hou W., Han T. (2015) The characterization of GmTIP, a root-specific gene from soybean, and the expression analysis of its promoter. Plant Cell Tiss Organ Cult. 121: 259.
Chong J (2002) Downregulation of a Pathogen-Responsive Tobacco UDP-Glc:Phenylpropanoid Glucosyltransferase Reduces Scopoletin Glucoside Accumulation, Enhances Oxidative Stress, and Weakens Virus Resistance. The Plant Cell Online 14: 1093-1107
Kuromori T, Hirayama T, Kiyosue Y, Takabe H, Mizukado S, Sakurai T, et al. (2001) A collection of 11 800 single-copy Ds transposon insertion lines in Arabidopsis. Plant J. 37:897-905.
Matsumoto S, Mizutani M, Sakata K, Shimizu B. (2012) Molecular cloning and functional analysis of the ortho-hydroxylases of p-coumaroyl coenzyme A/feruloyl coenzyme A involved in formation of umbelliferone and scopoletin in sweet potato, Ipomoea batatas (L.) Lam. Phytochemistry. 74, 49-57
McCarthy R L, Zhong R, Ye Z H (2011) Secondary wall NAC binding element (SNBE), a key cis-acting element required for target gene activation by secondary wall NAC master switches. Plant signaling & behavior 6: 1282-1285
Ohwaki, Y, & Sugahara, K. (1997). Active extrusion of protons and exudation of carboxylic acids in response to iron deficiency by roots of chickpea (Cicer arietinum L.). Plant and Soil, 189(1), 49-55.
Vanholme R, Storme V, Vanholme B, et al., 2012. A systems biology view of responses to lignin biosynthesis perturbations in Arabidopsis. Plant Cell 24, 3506-29.
Vanholme R, Morreel K, Darrah C, Oyarce P, Grabber J H, Ralph J, Boerjan W. 2012. New Phytol. 196(4):978-1000.
Rodriguez-Celma J, Lin W-D, Fu G-M, Abadla J, López-Millán A-F, Schmidt W, 2013. Mutually exclusive alterations in secondary metabolism are critical for the uptake of insoluble iron compounds by Arabidopsis and Medicago truncatula. Plant Physiol 162, 1473-85.
Schmid N B, Giehl R F, Doll S, et al., 2014. Feruloyl-CoA 6′-Hydroxylase-1-dependent coumarins mediate iron acquisition from alkaline substrates in Arabidopsis. Plant Physiol 164, 160-72.
Schmidt H, Gunther C, Weber M, et al., 2014. Metabolome analysis of Arabidopsis thaliana roots identifies a key metabolic pathway for iron acquisition. PloS one 9, e102444.
Sundaresan V., Springer P., Volpe T., Haward S., Jones J. D., Dean C., Ma H., Martienssen R. (1995). Patterns of gene action in plant development revealed by enhancer trap and gene trap transposable elements. Genes Dev. 9, 1797-1810
Sun H H, Wang L, Zhang B Q, Ma J H, Hettenhausen C, Cao G Y, Sun G L, Wu J Q, Wu J S (2014) Scopoletin is a phytoalexin against Altenaria alternata in wild tobacco dependent on jasmonate signalling. J Exp Bot 65: 4305-4315
Yang S.-M., Shim G. Y., Kim B.-G. and Ahn J.-H. (2015) Biological synthesis of coumarins in Escherichia coli. Microbial Cell Factories. 14:65

1. A chimeric gene construct comprising the following operably linked DNA elements:
a plant expressible promoter,
a DNA region encoding SEQ ID NO: 2 or a functional plant orthologue thereof; and
a 3′ end region comprising transcription termination and polyadenylation signals functioning in cells of a plant.

2. The chimeric gene construct of claim 1, wherein the DNA region is a multicistronic DNA region encoding SEQ ID NO: 2 or a functional plant orthologue thereof coupled to a DNA region encoding a feruloyl-CoA 6′-hydroxylase 1 or a functional plant orthologue thereof.

3. A recombinant vector comprising the chimeric gene construct of claim 1.

4. A plant, plant cell, or plant seed comprising the chimeric gene construct of claim 1.

5. A plant, plant cell or plant seed comprising
the chimeric gene construct of claim 1; and
a chimeric gene construct comprising the following operably linked DNA elements:
a plant expressible promoter,
a DNA region encoding a feruloyl-CoA 6′-hydroxylase 1 (F6′H1) or a functional plant orthologue thereof, and
a 3′ end region comprising transcription termination and polyadenylation signals functioning in cells of a plant.

6. A method of preventing iron deficiency chlorosis, increasing the production of coumarins, and/or increasing fungal resistance in a plant the method comprising:
expressing the DNA region of the chimeric gene construct of claim 1 in the plant.

7. (canceled)

8. (canceled)

9. A method for producing a plant with reduced iron deficiency chlorosis, increased coumarin production, increased fungal resistance, and/or alternative lignin monomers as compared to a corresponding wild-type plant, the method comprising:
introducing into a plant or transforming a plant with the chimeric gene of claim 1, and
selecting a plant with a stable expression of the chimeric gene.

10. (canceled)

11. (canceled)

12. (canceled)

13. The plant, plant cell or plant seed of claim 4, wherein the plant, plant cell or plant seed is a crop plant.

14. The plant, plant cell or plant seed of claim 4, wherein the plant, plant cell or plant seed is a tree.

15. The plant, plant cell, or plant seed of claim 5, wherein the plant, plant cell, or plant seed is a crop plant.

16. The plant, plant cell, or plant seed of claim 5, wherein the plant, plant cell, or plant seed is a tree plant.

17. The method according to claim 6, wherein the plant is a crop plant.

18. The method according to claim 6, wherein the plant is a tree.

19. The method according to claim 9, wherein the plant is a crop plant.

20. The method according to claim 9, wherein the plant is a tree.


SEQUENCE LISTING (16 sequences)

SEQ ID NO: 1 - DNA, 1356 bp, Arabidopsis thaliana
atggcgacacttgaaattaccgatatagccctggttcaaccttctcaccaaccactctccaacgaccaaactctctctctttcccatctcgacaatgataacaacctccacgtcagcttccgttacctccgcgtctactcctcctcctcttccaccgtcgccggagaaagcccctctgccgtcgtatccgcctctcttgccaccgctctcgttcactactaccctcttgctggctctctccgtcgctctgcctccgataaccgatttgaactactctgctccgctggtcaaagcgtgcctttagtcaacgctacagtgaactgtacgcttgagtcagtcgggtatttggatggacccgatccaggtttcgtcgagagattggtaccggatccgacccgggaggaaggaatggtcaatccttgtatcctccaggtcactatgtttcagtgtggtggttgggttctaggtgcatcgattcatcatgcgatctgcgatgggttaggtgcgagtctgttcttcaacgctatggcggaattagctcgcggagcgacaaagatttcgatcgaaccggtttgggacagagaacgtttacttggtccaagggagaagccttgggttggagctccagttcgtgacttcttgagcctggataaggactttgatccatatggacaagccattggagacgtcaaaagagactgcttctttgtgaccgacgattctttggatcaattgaaggctcaattactcgagaaatcgggtctgaatttcaccacattcgaagctcttggtgcttacatttggcgtgcaaaggtaagagctgcaaagactgaggaaaaggagaatgtgaaatttgtgtattcgataaatataaggagattgatgaatccacctttgcctaaaggctactggggaaatggatgtgtgccaatgtatgctcagatcaaagctggagaactcattgagcaaccaatctggaaaactgcagagctcataaaacagagcaaatccaatacgagtgatgaatatgtacgctcctttatcgacttccaagagctgcatcacaaagatggaatcaatgccggtacaggagtgaccggattcacggactggcgatacttggggcattccacgattgattttggatggggaggacctgtgacggttttgccactatcaaacaagttgcttggaagcatggaaccatgttttttcttgccatattctactgatgctgcagctggaagcaagaaagacagtgggtttaaggttttggtaaatctgcgcgaatctgcaatgcctgagtttaaagaggccatggataagttccacaaaggtgaatttgccctgtcttga

SEQ ID NO: 2 - protein, 451 aa, Arabidopsis thaliana
MATLEITDIALVQPSHQPLSNDQTLSLSHLDNDNNLHVSFRYLRVYSSSSSTVAGESPSAVVSASLATALVHYYPLAGSLRRSASDNRFELLCSAGQSVPLVNATVNCTLESVGYLDGPDPGFVERLVPDPTREEGMVNPCILQVTMFQCGGWVLGASIHHAICDGLGASLFFNAMAELARGATKISIEPVWDRERLLGPREKPWVGAPVRDFLSLDKDFDPYGQAIGDVKRDCFFVTDDSLDQLKAQLLEKSGLNFTTFEALGAYIWRAKVRAAKTEEKENVKFVYSINIRRLMNPPLPKGYWGNGCVPMYAQIKAGELIEQPIWKTAELIKQSKSNTSDEYVRSFIDFQELHHKDGINAGTGVTGFTDWRYLGHSTIDFGWGGPVTVLPLSNKLLGSMEPCFFLPYSTDAAAGSKKDSGFKVLVNLRESAMPEFKEAMDKFHKGEFALS

SEQ ID NO: 3 - DNA, 1086 bp, Arabidopsis thaliana
atggctccaacactcttgacaacccaattctcaaatccagctgaagtaaccgactttgtagtctacaaaggaaatggtgttaagggtttatcagaaacaggaatcaaagctcttccagaacaatacattcagccacttgaagaacgactcatcaacaaattcgtcaacgaaacagatgaagccattccagttatcgatatgtcgaaccctgatgaggacagagtcgctgaagctgtttgtgatgctgctgagaaatgggggttctttcaagtgatcaatcatggagttcctttggaagttcttgatgacgtcaaggctgcgactcacaagttcttcaatctccctgttgaagagaagcgcaagttcactaaagagaattcgctgtcgacgactgttaggtttgggacgagttttagtcctcttgcagagcaagcgcttgagtggaaagattatctcagcctcttctttgtctctgaagctgaagctgaacagttctggcctgatatctgcaggaatgaaacgttagagtacattaacaagtcaaagaagatggtgaggaggcttctagagtatttgggaaagaatctcaatgttaaagagcttgacgagacgaaagaatcactctttatgggctcgattcgagtcaaccttaactactaccccatctgccctaatccggacctaacagttggtgttggtcgccactcagacgtctcttctctcaccattctcttacaagaccagatcggtggtctacacgtgcgttctctggcttcagggaactgggttcacgtgcctccggttgctggatcttttgtgatcaacatcggagatgcgatgcagatcatgagcaatggtctgtacaagagcgtggagcatcgtgtcttagccaatggttacaataatagaatctctgttcctatctttgtgaacccaaaaccagagtcagttattggtcctctacctgaggtgattgcaaacggagaggaaccgatttacagagacgtcctgtactctgattacgtcaagtatttcttcaggaaggcacacgatggaaagaaaaccgtcgattacgccaagatctga

SEQ ID NO: 4 - protein, 361 aa, Arabidopsis thaliana
MAPTLLTTQFSNPAEVTDFVVYKGNGVKGLSETGIKALPEQYIQPLEERLINKFVNETDEAIPVIDMSNPDEDRVAEAVCDAAEKWGFFQVINHGVPLEVLDDVKAATHKFFNLPVEEKRKFTKENSLSTTVRFGTSFSPLAEQALEWKDYLSLFFVSEAEAEQFWPDICRNETLEYINKSKKMVRRLLEYLGKNLNVKELDETKESLFMGSIRVNLNYYPICPNPDLTVGVGRHSDVSSLTILLQDQIGGLHVRSLASGNWVHVPPVAGSFVINIGDAMQIMSNGLYKSVEHRVLANGYNNRISVPIFVNPKPESVIGPLPEVIANGEEPIYRDVLYSDYVKYFFRKAHDGKKTVDYAKI

SEQ ID NO: 5 - DNA, 2493 bp, Artificial Sequence
  bicistronic construct F6′H1T2ACOSY
atggctccaacactcttgacaacccaattctcaaatccagctgaagtaaccgactttgtagtctacaaaggaaatggtgttaagggtttatcagaaacaggaatcaaagctcttccagaacaatacattcagccacttgaagaacgactcatcaacaaattcgtcaacgaaacagatgaagccattccagttatcgatatgtcgaaccctgatgaggacagagtcgctgaagctgtttgtgatgctgctgagaaatgggggttctttcaagtgatcaatcatggagttcctttggaagttcttgatgacgtcaaggctgcgactcacaagttcttcaatctccctgttgaagagaagcgcaagttcactaaagagaattcgctgtcgacgactgttaggtttgggacgagttttagtcctcttgcagagcaagcgcttgagtggaaagattatctcagcctcttctttgtctctgaagctgaagctgaacagttctggcctgatatctgcaggaatgaaacgttagagtacattaacaagtcaaagaagatggtgaggaggcttctagagtatttgggaaagaatctcaatgttaaagagcttgacgagacgaaagaatcactctttatgggctcgattcgagtcaaccttaactactaccccatctgccctaatccggacctaacagttggtgttggtcgccactcagacgtctcttctctcaccattctcttacaagaccagatcggtggtctacacgtgcgttctctggcttcagggaactgggttcacgtgcctccggttgctggatcttttgtgatcaacatcggagatgcgatgcagatcatgagcaatggtctgtacaagagcgtggagcatcgtgtcttagccaatggttacaataatagaatctctgttcctatctttgtgaacccaaaaccagagtcagttattggtcctctacctgaggtgattgcaaacggagaggaaccgatttacagagacgtcctgtactctgattacgtcaagtatttcttcaggaaggcacacgatggaaagaaaaccgtcgattacgccaagatcgagggcagaggaagtctgctaacatgcggtgacgtcgaggagaatcctggcccaatggcgacacttgaaattaccgatatagccctggttcaaccttctcaccaaccactctccaacgaccaaactctctctctttcccatctcgacaatgataacaacctccacgtcagcttccgttacctccgcgtctactcctcctcctcttccaccgtcgccggagaaagcccctctgccgtcgtatccgcctctcttgccaccgctctcgttcactactaccctcttgctggctctctccgtcgctctgcctccgataaccgatttgaactactctgctccgctggtcaaagcgtgcctttagtcaacgctacagtgaactgtacgcttgagtcagtcgggtatttggatggacccgatccaggtttcgtcgagagattggtaccggatccgacccgggaggaaggaatggtcaatccttgtatcctccaggtcactatgtttcagtgtggtggttgggttctaggtgcatcgattcatcatgcgatctgcgatgggttaggtgcgagtctgttcttcaacgctatggcggaattagctcgcggagcgacaaagatttcgatcgaaccggtttgggacagagaacgtttacttggtccaagggagaagccttgggttggagctccagttcgtgacttcttgagcctggataaggactttgatccatatggacaagccattggagacgtcaaaagagactgcttctttgtgaccgacgattctttggatcaattgaaggctcaattactcgagaaatcgggtctgaatttcaccacattcgaagctcttggtgcttacatttggcgtgcaaaggtaagagctgcaaagactgaggaaaaggagaatgtgaaatttgtgtattcgataaatataaggagattgatgaatccacctttgcctaaaggctactggggaaatggatgtgtgccaatgtatgctcagatcaaagctggagaactcattgagcaaccaatctggaaaactgcagagctcataaaacagagcaaatccaatacgagtgatgaatatgtacgctcctttatcgacttccaagagctgcatcacaaagatggaatcaatgccggtacaggagtgaccggattcacggactggcgatacttggggcattccacgattgattttggatggggaggacctgtgacggttttgccactatcaaacaagttgcttggaagcatggaaccatgttttttcttgccatattctactgatgctgcagctggaagcaagaaagacagtgggtttaaggttttggtaaatctgcgcgaatctgcaatgcctgagtttaaagaggccatggataagttccacaaaggtgaatttgccctgtcttga

SEQ ID NO: 6 - DNA, 1347 bp, Populus balsamifera subsp. trichocarpa
atggaagaaattcacatcaaagaaaccattcccattcgcccttctacgccccctttctctcaagaccatacactccctctctcccaccttgacaccgaccgcaaccttaatgtgacatttcgctaccttcgtgtctacgttaacaccaccaccagcaatggtggccatcctttcaacgtcatcgctgctgcgctttcctctgctcttgttcactactaccctcttgcagccactctccgccgaggccaggtggatgaccgtctggagctgttttgcactcgggaccatttaggtgtgcctcttatcaatgcaactgtaaactgcacgttggaaaaactgaactaccttgatgactcggacccgaattttctggacgggttagttcctgacccggatcaagattatgggctggctaacccgtgtgttctccaagtcacggtgtttgagtgtggtggatggacattaggtgctgccatacaccatggtttgtgtgatgggcttggggcaacccagttttttaatgttatggcagagttggcacgtggtgtgggtcggatttcagctaatccagtttgggaccgggcgcgcttgttgggtccgagagacccgccccgagctgagggagtggtgagggagtttttggggttggagaaagggtctgagccttacggtcaggtggttggtgaggttgtgagggagtgctttcctgtgaaggatgagtggttggagaagttcaagaaggtgttgtttgagaagagtggctctagctttaccacgtttgaagccttgggtgcattcatatggcgggcaaaggttaaagcctcaggggttccaggtgatgagaatgtgaagttcgcctattcaatcaatatacgaaaactagtaaagccaccattacctgctggctattggggcaatggttgtgttccgatgtatgctcaactctgtgccagagagctgatagagcaaccagtttggaaaacagctgagctgattaaaaagagcaaaatcaacgcaaccgatgagtacgtccgctcattcattgattttcaagaactacattatggagatggcattacagcaggaaacagagtgagtgggttcacagattggaggcacctgggacattcaactgttgattttgggtggggaggtccggtcactgtcttgccactttcaagaaaactactcggaagtgttgtgccgtgctttttcttgccttattcttctgcaaatgcaggcaagaaggatgggttcaaggtgctggtaaccttgcaagaaacacacatgcctgccttcaagaaagagatggagaaatttagcagacaagattttgacttgtcttga

SEQ ID NO: 7 - protein, 448 aa, Populus balsamifera subsp. trichocarpa
MEEIHIKETIPIRPSTPPFSQDHTLPLSHLDTDRNLNVTFRYLRVYVNTTTSNGGHPFNVIAAALSSALVHYYPLAATLRRGQVDDRLELFCTRDHLGVPLINATVNCTLEKLNYLDDSDPNFLDGLVPDPDQDYGLANPCVLQVTVFECGGWTLGAAIHHGLCDGLGATQFFNVMAELARGVGRISANPVWDRARLLGPRDPPRAEGVVREFLGLEKGSEPYGQVVGEVVRECFPVKDEWLEKFKKVLFEKSGSSFTTFEALGAFIWRAKVKASGVPGDENVKFAYSINIRKLVKPPLPAGYWGNGCVPMYAQLCARELIEQPVWKTAELIKKSKINATDEYVRSFIDFQELHYGDGITAGNRVSGFTDWRHLGHSTVDFGWGGPVTVLPLSRKLLGSVVPCFFLPYSSANAGKKDGFKVLVTLQETHMPAFKKEMEKFSRQDFDLS

SEQ ID NO: 8 - DNA, 1359 bp, Glycine max
atgcaaaggatcaaaacctcagaacgcactttgatcttcccttcccaccctccttttctccaagaccaccccttccctctctcccacctcgacactgatcccaacctccaccttaccttccgctacctccgcgcgtacacctcaacaacaacaacaacctccctcgaccccttccacgtcatctcctcctccctctcccacgccctcccccacttctaccccctcaccgccaccctccgccgccaacaaacctccccccaccgcctccaactctggtgcgtcgccggccagggcatccccctcatccgcgccaccgcggacttcaccctcgagtccgtgaacttcctcgacaacccggcctcgagcttcttggagcagttagtgcccgacccgggacccgaggaggggatggagcacccgtgcatgctccaggtgacggtgttcgcgtgcgggggattcaccctcggcgcggcgatgcaccacgcgctctgcgacggcatgggcgggacgctgttcttcaatgcggtggcggagctggcgcgtggggcgacccggataacgttggacccggtttgggaccgtgcgaggttgctgggtcccagggacccgcccctggtggattcgccgttgattggggagtttctgcgtttggagaagggagttttgccgtaccaacagagtgttggtggggtcgcgagagagtgctttcacgtgaaggatgagtgcttggacaatttcaagaggaccttgttggagcaatctgggttgaacttcaccgtttttgaggctcttggtgcctacatctggagggctaaggtcagggcctcgggaatccaggctgatgaaaaggtgaagtttgcatactcaattaacatacggagactggtaaagccaccactgcctggtggctattggggtaatggttgtgtgccaatgtacgtacaacttagtgccaaagatttgatagagaaacccgtttgcgaaaccgcagagctaataaaaaagagtaaaagcaatgtcactgatgagtatgttaagtcctacatcgattatcaggagctgcattttgctgatggaatcactgcgggaaaagaggttagtgggttcacggattggaggcacttgggccattcaactgtggactttgggtggggtggcccagttactgttttgccccttggaaggaacttacttgggagtgttgagccttgcttttttttgccttattcaacagccacttcagagaagaaagaggggttcaaggttttggtgactttgagagaggctgcgttgcctgctttcagagaagacatgaaagtgttttgcaatagccaagagcagtggctgagccacatatag

SEQ ID NO: 9 - protein, 452 aa, Glycine max
MQRIKTSERTLIFPSHPPFLQDHPFPLSHLDTDPNLHLTFRYLRAYTSTTTTTSLDPFHVISSSLSHALPHFYPLTATLRRQQTSPHRLQLWCVAGQGIPLIRATADFTLESVNFLDNPASSFLEQLVPDPGPEEGMEHPCMLQVTVFACGGFTLGAAMHHALCDGMGGTLFFNAVAELARGATRITLDPVWDRARLLGPRDPPLVDSPLIGEFLRLEKGVLPYQQSVGGVARECFHVKDECLDNFKRTLLEQSGLNFTVFEALGAYIWRAKVRASGIQADEKVKFAYSINIRRLVKPPLPGGYWGNGCVPMYVQLSAKDLIEKPVCETAELIKKSKSNVTDEYVKSYIDYQELHFADGITAGKEVSGFTDWRHLGHSTVDFGWGGPVTVLPLGRNLLGSVEPCFFLPYSTATSEKKEGFKVLVTLREAALPAFREDMKVFCNSQEQWLSHI

SEQ ID NO: 10 - DNA, 1350 bp, Medicago truncatula
atgttgaaaatcaaagaacaaactctcatttttccttctcacacccccttccttgaagaccacaccctccctctctcccacctcgacatcgaccgcaacctcaatacaaccatacgctacctccgcgcttacacagccaccaccacccaccaccatgatcccttcaccgtcatctcctcctccctctcaaaaactctccctcattactacccactcgccgccacactccgctaccacaaccaccgcctcgaactcttctgctccaaaagccaagacagcgttcctctcatccacgccaccgtagacatcactctcgaatccgtaaactacctcgacgacgacccatcttcccattttgttgaacagttagtacccgacccaaaaccggaagaaggattaaaccatccatgcatgcttcaactcactgtttacaagtgcggtgggttcactctcggtgcagcaattcaccattcactttgtgatggaatgggtgggacgctttttttcaacacgatggcggagttggctcgtggcggggaacatatcatggtggagccagtgtgggatagagagaagttgttgggtccaagggatgtgccacgagtggattcggcgttggtaagagagtttttgagtttggataaagagtttttggtgtatgaagaaggtgatggtggtgttgtgagagagtgttttcatgtgaaggatgagtgtttggaagagtttaagagatctttgtttgatcaatgtgggttcaagttcaccacttttgaagctttaggtgcttgcatttggaggtccaaggtaaaggcctcaaaagtggaggataatgagaaggtgaagtttgcatactcaatcaatatacgtagactagtaaagccatcactacctgctggatattggggaaatggttgtgttccaatgtatgttcaattgagtgctaaagatttgatagaacaacccatttgggaaacagcagaactaataagaaagagtaaaaccaatgtcactgatgagtatgttcgctctttcattgattatcaacatttgcattatgctgatgggatcacagcaggaaaatgggtgagtggtttcactgattggagacacttgggccattcaactgtggattttggttggggaggtcctgttactgttttgccacttggtagaaacctgcttgggagtgttgaaccttgttactttttgccttattcaatagccagtgctgacaaaaagaatgggtttaaggttttggtgaatttgagtgaggtagctttgcctgcttttacagaagatatgcaaatgtttgctggtagtcgagagttgttacccgagtctcgcatttga

SEQ ID NO: 11 - protein, 449 aa, Medicago truncatula
MLKIKEQTLIFPSHTPFLEDHTLPLSHLDIDRNLNTTIRYLRAYTATTTHHHDPFTVISSSLSKTLPHYYPLAATLRYHNHRLELFCSKSQDSVPLIHATVDITLESVNYLDDDPSSHFVEQLVPDPKPEEGLNHPCMLQLTVYKCGGFTLGAAIHHSLCDGMGGTLFFNTMAELARGGEHIMVEPVWDREKLLGPRDVPRVDSALVREFLSLDKEFLVYEEGDGGVVRECFHVKDECLEEFKRSLFDQCGFKFTTFEALGACIWRSKVKASKVEDNEKVKFAYSINIRRLVKPSLPAGYWGNGCVPMYVQLSAKDLIEQPIWETAELIRKSKTNVTDEYVRSFIDYQHLHYADGITAGKWVSGFTDWRHLGHSTVDFGWGGPVTVLPLGRNLLGSVEPCYFLPYSIASADKKNGFKVLVNLSEVALPAFTEDMQMFAGSRELLPESRI

SEQ ID NO: 12 - DNA, 1392 bp, Oryza sativa ssp. japonica
atggagtcgtcgccgccgccgccgccgcagatgcgtgtgcgcgtcatggagaccgtgcacctgcgtccgcctccggcggatgacgcggcgtcgttcgcgctgtccgggctggacacggaccgcaacgtgctcgacgtgacgttccgcacgctgcgcttcttcccgccgccgtcgctcgagctcgacccgctcgccgtcctcccgcgcgcgttcgccgccgccctgggcatgttcgtcccgctcgccgggaggattggggacggagggcgcgtcgtctggtcggccgccgacgccgtgccccttgttctcgccgcggcggacgacgtgtcggtggccgacgtcgacaccgacagcccgggctccgatttgctggagcggctcgtgccgggggacggcgacggcgacggcgtggcggggtcaccggcgctcgcgctccaggttacgcggttcgcctgcggcggcgtcgcgctggggatgcgggtggcgcacgcgctctgcgacggcgccggcgccaccaagttcctctccgccgcggcgcggttcgcgcgcggggcgcaggagccggcggcggtggcgccggtgtgggagcgggaggaccgtctcggcccgaggcgtccgccgcgcgtggtgaagccgttcgaacgcgtcctctcgctcgacgacgccgccgccgcggtgcacggaccgtacggcgcggccggcgacgcacaaggacagatcgcgagggagtgcttccacgtgagcgacgcgcgcgtggaggagctcagggcgcagctcgccggcgaggccggcatcaagctcacaacgttcgagttcctcgccgcgttcatctggcgcgccaggacgaaagccagaaggacgagccccgacgaggtcgtgaagatggtgtactccatgaacatcagcaagctcctcacgccgcctctcccggacggctactggggcaacgtgtgcgtcccagtgtacgtcgccctcaccgccggcgagctcgtcgcccagccgctcgcggacacagccgccatggtcaagaagagcaagcaggaggtggacgacgagtacgtccgatcctacatcgacttccacgagctccaccgcggcggcggcgtcacggcggggcgcggcgtgagcgcgttcaccgactggcgccgcctcggccactcggaggtggacttcgggtggggctcgccggcggccgtgctgccgctctcgtggaggctgctcgggagcacggagccgtgcttcttcgtgccgtacggcgcagccgacgagaggcggcggcgcgggttcaaggtgttcgtcgcggtgccggcgatggcgacgcactgcttcagagaggagatgcaggagctatcgttgcaacgccattgcctgcgttcgaaagagaagctgtaa

SEQ ID NO: 13 - protein, 463 aa, Oryza sativa ssp. japonica
MESSPPPPPQMRVRVMETVHLRPPPADDAASFALSGLDTDRNVLDVTFRTLRFFPPPSLELDPLAVLPRAFAAALGMFVPLAGRIGDGGRVVWSAADAVPLVLAAADDVSVADVDTDSPGSDLLERLVPGDGDGDGVAGSPALALQVTRFACGGVALGMRVAHALCDGAGATKFLSAAARFARGAQEPAAVAPVWEREDRLGPRRPPRVVKPFERVLSLDDAAAAVHGPYGAAGDAQGQIARECFHVSDARVEELRAQLAGEAGIKLTTFEFLAAFIWRARTKARRTSPDEVVKMVYSMNISKLLTPPLPDGYWGNVCVPVYVALTAGELVAQPLADTAAMVKKSKQEVDDEYVRSYIDFHELHRGGGVTAGRGVSAFTDWRRLGHSEVDFGWGSPAAVLPLSWRLLGSTEPCFFVPYGAADERRRRGFKVFVAVPAMATHCFREEMQELSLQRHCLRSKEKL

SEQ ID NO: 14 - DNA, 1712 bp, Arabidopsis thaliana
aaataatgattttattttgactgatagtgacctgttcgttgcaacaaattgataagcaatgcttttttataatgccaactttgtatagaaaagttgtaactgcaacgaagtgtaccaacaacttgactaggattctaagttcttttatgtataggatgtctatattaaactaccatgactaacatatatatagtagttccatatgctcgataaactatgatagatcaacaattttaaacatatagtttaacactatttatttgttcaacgtcaatagtttatagttacgcatgcgctcggcttagatttggtccccaacagtcgaaattgtcaaataatataaaataaaagtttcattgttaggattcatttattcttcgggtggttattgtaataaaaggcaaaagaaaaagaagaacaaaattcacaagtaaaaaaaaagataacatcattcttttagtcgacaaaaaaaaaaaaaaatcaaaaagatttattcagtactacagtttaatattgttttgacttttttctttttctttatattatctgaaaattctagactgcagctgaaacatgtgatatggattaaaggcgtatccagtatccacataaagaggagtggtgtcgctcacccagtcacccttgttacttgttagatagcattaatacatttgtaagcaacagcttatctgatagacatgtcttaattgggaaatatgctctaagatgatacaaccatggttccaactgttgaccaccatagctgataacatgttgattacattttttcttttcagttataacgattacttttttggggaaattattgatataatatgattcattggatgatccgatatcatgcatataaagttgtatctcgtgaaacacgagatagtattatactccattctttcattatcggagtatgtttaaaatttgaaaacaaatacagacacggaccgtggtctttaccttcagaaaaaaaaagagaaaaaaaaaacaatccactgtttattataggagttgtagaaaatcgggcaacgatattcgatatgagttattattagggccttattattatatggtattactggatattactaaataaaataatcatataaatttcacattttaatatacactcgttggacacgcggaatattatatgttctaaatgttaaaaaaatcaaaagaaatacaacgatcgacggatctagagtctagaccatgcaaataaatcatcctatttaaatataataactgtgcatatagtttagtcaaataaaaaggtaaagaaacaatatacaacctataacgtcaatatccatgtacgtagtaataattaggatatgacagaaaacacgatatcttgatatatacaaaatgaaaacttaaaaattgattaatatggcctggctgggtatattattataaaaacataaagagagatcaataattgattcgaagatcactatataaagaacgtcttcgatatgtaaaagaaccatcctaaacattttttcttgaataaaatcagaattacaaacaaaatcaagtttgtacaaaaaagttgaacgagaaacgtaaaatgatataaatatcaatatattaaattagattttgcataaaaaacagactacataatactgtaaaacacaacatatgcagtcactatgaatcaactacttagatggtattagtgacctgta

SEQ ID NO: 15 - DNA, 1987 bp, Arabidopsis thaliana
atgaagccatcctctacctcggaaaaacttgttgcgagaagaagacatgcgatggcatggatgcttggatctttgacattgatgacactcttctctcaaccattccttaccacaagagcaacggttgtttcgggtaaataaactaaacttaaccatatacattagccttgattcggtttttggtttgatttatggatattaaagatccgaattatatttgaacaaaaaaaaatgattatgtcacataaaaaaaaattggcttgaattttggtttagatgggtttaaatgtctacctctaatcatttcatttgttttctggttagctttaattcggtttagaatgaaaccgggattgacatgttacattgatttgaaacagtggtgagcaactgaacacgaccaagttcgaggaatggcaaaattcgggcaaggcaccagcggttccacacatggtgaagttgtaccatgagatcagagagagaggtttcaagatctttttgatctcttctcgtaaagagtatctcagatctgccaccgtcgaaaatcttattgaagccggttaccacagctggtctaacctccttctgaggttcgaatcatatttaataaccgcattaaaccgaaatttaaattctaatttcaccaaatcaaaaagtaaaactagaacacttcagataaattttgtcgttctgttgacttcatttattctctaaacacaaagaactatagaccataatcgaaataaaaaccctaaaaaccaaatttatctatttaaaacaaacattagctatttgagtttcttttaggtaagttatttaaggttttggagactttaagatgttttcagcatttatggttgtgtcattaatttgtttagtttagtaaagaaagaaaagatagtaattaaagagttggttgtgaaatcatatttaaaacattaataggtatttatgtctaatttggggacaaaatagtggaattctttatcatatctagctagttcttatcgagtttgaactcgggttatgattatgttacatgcattggtccatataaatctatgagcaatcaatataattcgagcattttggtataacataatgagccaagtataacaaaagtatcaaacctatgcaggggagaagatgatgaaaagaagagtgtgagccaatacaaagcagatttgaggacatggcttacaagtcttgggtacagagtttggggagtgatgggtgcacaatggaacagcttctctggttgtccagttcccaagagaaccttcaagctccctaactccatctactatgtcgcctgattaaatcttatttactaacaaaacaataagatcagagtttcattctgattcttgagtcttttttttctctctccctcttttcatttctggtttatataaccaattcaaatgcttatgatccatgcatgaaccatgatcatctttgtgtttttttttccttctgtattaccattttgggcctttgtgaaattgattttgggcttttgttatataatctcctctttctctttctctacctgattggattcaagaacatagccagatttggtaaagtttataagatacaaaatattaagtaagactaaagtagaaatacataataacttgaaagctactctaagttatacaaattctaaagaactcaaaagaataacaaacagtagaagttggaagctcaagcaattaaattatataaaaacactaactacactgagctgtctccttcttccaccaaatcttgttgctgtctcttgaagctttcttatgacacaaaccttagacccaatttcactcacagtttggtacaacctcagttttcttcacaacaaattcaaacatcttacccttatattacctctttatctcttcaatcatcaaaacacatagtcacatacatttctctaccccaccttctgctctgcttccgagagctcagtgtacctcgccgctag

SEQ ID NO: 16 - DNA, 1119 bp, Cauliflower mosaic virus
atttaggtgacactatagaatactcaagctatgcatccaacgcgttgggagctctcccatatggtcgagatctcctttgccccggagatcaccatggacgactttctctatctctacgatctaggaagaaagttcgacggagaaggtgacgataccatgttcaccaccgataatgagaagattagcctcttcaatttcagaaagaatgctgacccacagatggttagagaggcctacgcggcaggtctcatcaagacgatctacccgagtaataatctccaggagatcaaataccttcccaagaaggttaaagatgcagtcaaaagattcaggactaactgcatcaagaacacagagaaagatatatttctcaagatcagaagtactattccagtatggacgattcaaggcttgcttcataaaccaaggcaagtaatagagattggagtctctaagaaagtagttcctactgaatcaaaggccatggagtcaaaaattcagatcgaggatctaacagaactcgccgtgaagactggcgaacagttcatacagagtcttttacgactcaatgacaagaagaaaatcttcgtcaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggactgcaggacgatccgtatttttacaacaattaccacaacaaaacaaacaacaaacaacattacaatttactattctagtcgacctgcaggcggccgcactagtgatatca